(19) United States
(12) Patent Application Publication
(10) Pub. No.: US 2010/0087546 A1
(43) Pub. Date: Apr. 8, 2010

(54) USE OF DIMETHYL SULFONE (MSM) TO REDUCE HOMOCYSTEINE LEVELS

(75) Inventor: Jeremy E. Appleton, Portland, OR (US)

Correspondence Address: KNOBBE MARTENS OLSON & BEAR LLP, 2040 MAIN STREET, FOURTEENTH FLOOR, IRVINE, CA, 92614, US

(73) Assignee: BIOGENIC INNOVATIONS, LLC, Vancouver, WA (US)

(21) Appl. No.: 12/562,942

(22) Filed: Sep. 18, 2009

Related U.S. Application Data
(63) Continuation-in-part of application No. 11/407,715, filed on Apr. 20, 2006, now abandoned.
(60) Provisional application No. 60/673,051, filed on Apr. 20, 2005.

(51) Int. Cl. A61K 31/10; A61P 43/00

(52) U.S. Cl. 514/711

(57) ABSTRACT

A method for reducing elevated levels of homocysteine in a subject comprising the steps of measuring the level of homocysteine in a subject; detecting an elevated level of homocysteine in the subject; and responsive to said step of detecting, administering to said subject a nutritional supplement comprising dimethyl sulfone (MSM).

### RELATED APPLICATIONS

This application is a continuation-in-part of U.S. application Ser. No. 11/407,715, filed Apr. 20, 2006, which claims benefit to U.S. Provisional Application No. 60/673,051, filed Apr. 20, 2005, the entire contents of which are hereby expressly incorporated by reference.

### FIELD OF THE INVENTION

Embodiments of the invention relate to the use of dimethyl sulfone (also called methylsulfonylmethane or MSM). Methods for using dimethyl sulfone for supporting normal homocysteine levels and for preventing and treating elevated homocysteine levels in the blood are also provided. Several embodiments relate to the administration of dimethyl sulfone by multiple routes, including oral, intravenous, topical, and other routes to reduce homocysteine and to maintain normal homocysteine levels.

Embodiments of the invention also relate to methods for using dimethyl sulfone in combination with other nutritional ingredients, dietary supplements, excipients, foods, beverages, food additives, and drugs for maintenance of desired homocysteine levels.

### BACKGROUND

Homocysteine is an amino acid formed in the body. It is created by the breakdown of another amino acid, methionine. High dietary consumption of methionine, which can be found in meats and dairy products, can result in the overproduction of homocysteine.

Homocysteine appears to be a nerve and vessel toxin, promoting mortality and disease. If the right cofactors are present, homocysteine will eventually convert to cysteine and other beneficial compounds. If the cofactors are lacking, homocysteine will build up to toxic levels.

FIG. 1 shows that homocysteine is metabolized in the body through one of two possible pathways—remethylation or transsulfuration. Remethylation is a process that utilizes folate, vitamin B-12 or betaine (trimethylglycine) to convert homocysteine back to methionine. Alternately, transsulfuration utilizes vitamin B6, pyridoxal-5-phosphate, to catabolize excess homocysteine into a number of metabolites for eventual excretion from the body.

Normal homocysteine levels are from 2.2 to 13.2 μmol. Loehrer, et al, Influence of oral S-adenosylmethionine on plasma 5-methyltetrahydrofolate, S-adenosylhomocysteine, homocysteine, and methionine in healthy humans. J Pharmacol Exp Ther., 1997 August; 282(2):845-50. Levels of homocysteine in typical Western populations are about 12 μmol. Lowering blood homocysteine with folic acid based supplements: meta-analysis of randomized trials. Homocysteine Lowering Trialists' Collaboration, BMJ. 1998 Mar. 21; 316(7135):894-8. Although this is “normal,” it is not necessarily healthy.

Research suggests that elevated serum levels of homocysteine are a major cause of cardiovascular disease and cerebrovascular disease. Cardiovascular disease includes ischemic heart disease (heart attack), coronary artery disease (plaque obstruction of the coronary arteries to the heart), and stroke. Elevated homocysteine levels are believed to damage coronary arteries or make it easier for platelets to clump together and form a clot. Studies have shown that high serum homocysteine-related blood vessel damage may account for up to 20% of heart attacks, 40% of strokes, and 60% of peripheral venous occlusions in the United States. Further, a meta-analysis conducted in 2002 concluded that a 5 μmol/l increase in homocysteine increased the risk of cardiovascular disease by 23% and of stroke by 42%.

Increasing evidence suggests that homocysteine is also associated with cognitive impairment and Alzheimer's disease. Homocysteine is, in fact, toxic to the medulloblastoma cells of the brain. This cell type may be involved in the degenerative processes of Alzheimer's and Parkinson's. People with elevated levels of homocysteine have nearly double the risk of developing Alzheimer's disease, according to a report from researchers at Boston University. The findings, which come from the long-running Framingham Study, are the first to tie homocysteine levels measured several years before with later diagnosis of Alzheimer's and other dementias.

Elevated homocysteine levels have also been associated with cognitive impairment; depression; chronic fatigue syndrome; rheumatoid arthritis; adverse outcomes in pregnancy, including premature births, preeclampsia, stillbirths, and birth defects (e.g., spina bifida, other neural tube defects, congenital heart defects); spontaneous abortion (miscarriage); schizophrenia; multiple sclerosis; osteoporosis; gastritis; renal diseases including renal failure, renal transplant, and uremia; oxidative stress; inflammation; elevated inflammatory mediators; eye disorders including nonarteritic anterior ischemic optic neuropathy and retinal venous occlusive disease; and cancer.

Oral vitamin formulations combining vitamin B-12, folic acid, and vitamin B-6 have traditionally been used in the treatment of elevated serum levels of homocysteine. However, many people suffer from conditions caused or exacerbated by, or associated with, elevated homocysteine levels and do not receive treatment since the symptoms of these diseases are not easily recognized. Thus, by the time a person finally suffers recognizable symptoms of one of these diseases or are diagnosed, the severity of the disease may have become life-threatening.

### SUMMARY

Several embodiments of the invention relate to the maintenance of healthy physiological levels of homocysteine. In some embodiments, the invention provides a dietary supplement and a method of using same for controlling and maintaining desired serum levels of homocysteine.

In another embodiment, the invention comprises a dietary supplement and a method of using same for treating or preventing diseases caused or exacerbated by elevated levels of homocysteine. Such diseases include, but are not limited to, cardiovascular diseases, cerebrovascular diseases, arthritis (such as rheumatoid arthritis or osteoarthritis), neurodegenerative diseases (such as Alzheimer's disease), adverse outcomes in pregnancy, renal diseases, eye disorders, and cancer.

In several embodiments, methods for reducing elevated levels of homocysteine in a human or non-human mammal is provided. In one embodiment, the method comprises measuring a homocysteine level in a subject, detecting an elevated level of homocysteine in the subject (e.g., person or non-human mammal), and responsive to the step of detecting, administering dimethyl sulfone to the subject. The dimethyl sulfone is administered in a daily dosage of dimethyl sulfone, wherein the daily dosage of dimethyl sulfone is greater than 3 grams, and wherein dimethyl sulfone reduces the elevated level of homocysteine in the subject. In one embodiment, dimethyl sulfone is administered in a daily dosage between 6 grams to 10 grams. Veterinary applications are contemplated in several embodiments.

The daily dosage dimethyl sulfone may be administered in a single dose or multiple dosages throughout the day. Oral or intravenous administration is provided according to some embodiments. Other routes of administration may also be used.

The administration of dimethyl sulfone, in some embodiments, reduces the elevated level of homocysteine in the subject by an amount sufficient to reduce pain in the subject.

In one embodiment, dimethyl sulfone reduces the elevated level of homocysteine in the subject by an amount sufficient to improve physical function in the subject. Homocysteine levels in urine, serum, plasma and/or other biological tissue is decreased following administration of dimethyl sulfone in several embodiments.

In one embodiment, dimethyl sulfone reduces the elevated level of homocysteine in the subject by at least 10%, by 25-50%, or 50%-100%. In other embodiments, dimethyl sulfone reduces the elevated level of homocysteine in the subject by at least two-, three-, four-, five- or ten-fold.

In one embodiment of the invention, a method of reducing pain or improving physical function in a subject having osteoarthritis and elevated homocysteine is provided. In one embodiment, the method comprises identifying subjects with osteoarthritis, measuring a homocysteine level in a subject having osteoarthritis, detecting an elevated level of homocysteine in the subject, and responsive to the step of detecting, administering dimethyl sulfone to the subject. The dimethyl sulfone is administered in a daily dosage of dimethyl sulfone, wherein the daily dosage of dimethyl sulfone is greater than 3 grams, and wherein the dimethyl sulfone reduces the elevated level of homocysteine in the subject. In one embodiment, dimethyl sulfone is administered in a daily dosage between 6 grams to 10 grams.

In any of the embodiments described herein, one, two, three or more additional compounds may be administered with dimethyl sulfone. Such additional compounds may be provided in a formulation together with dimethyl sulfone, or may be administered separately. One or more of the additional compounds may maintain or reduce homocysteine levels. Additional compounds may be metabolically related to dimethyl sulfone in some embodiments.

According to several embodiments herein, a therapeutically effective amount of dimethyl sulfone is administered. For example, in some embodiments, about 500 mg, 1 g, 1.5 g, 2 g, 2.5 g, 3 g, 3.5 g, 4 g, 4.5 g, 5 g, 5.5 g, 6 g, 6.5 g, 7 g, 7.5 g, and 8 g is provided as a therapeutically effective amount Doses of dimethyl sulfone are provided in single dose forms to be administered periodically (e., daily, weekly, or monthly). Twice daily doses are also provided. Pills, tablets, powders, capsules, gels, liquids, and inhalants comprising dimethyl sulfone (alone or in combination with other ingredients) are provided according to several embodiments herein. In several embodiments, dimethyl sulfone dosages are provided in levels not less than 3 g per day for at least 4 weeks, 6 weeks, 8 weeks, 10 weeks, 12 weeks, 16 weeks, 24 weeks, 6 months and 1 year. In some embodiments, the reduction in homocysteine occurs only while dimethyl sulfone is administered to the subject and continuous treatment with dimethyl sulfone is provided. In other embodiments, dimethyl sulfone reduces homocysteine to healthy levels and is discontinued (or amounts administered are reduced) once a desired target homocysteine level is reached.

In some embodiments, a formulation of dimethyl sulfone in combination with at least one other ingredient having homocysteine controlling properties. For example, in several embodiments, dimethyl sulfone is provided in combination with one or more of the following: vitamin B6, vitamin B12, folic acid, and riboflavin. In one embodiment, a composition consisting only of dimethyl sulfone is provided. In another embodiment, a composition consisting essentially of dimethyl sulfone is provided. In a further embodiment, a composition comprising dimethyl sulfone is provided. In another embodiment, cysteine is administered with dimethyl sulfone.

The methods and compositions according to several embodiments herein provide an effective and convenient method for controlling and maintaining homocysteine levels. In some embodiments, desired homocysteine levels are maintained. In other embodiments, homocysteine levels are reduced to a desired level.

In one embodiment, a composition and method for preventing or treating or preventing vascular disease is provided. In one embodiment, a formulation consisting, consisting essentially of or comprising dimethyl sulfone alone or in combination with nutrients, nutritional ingredients, nutraceuticals and/or drugs intended to alter homocysteine is provided.

In a further embodiments, dimethyl sulfone is provided in combination with one or more excipients.

In several embodiments, periodic administration of a therapeutically effective amount of dimethyl sulfone is provided for the prevention or treatment of the diseases described herein.

In several embodiments, the beneficial effects of dimethyl sulfone include the promotion and maintenance of desired homocysteine levels in the body. Another beneficial effect of several embodiments includes the ability to promote and maintain health. More particularly, many people suffer from conditions caused or exacerbated by, or associated with, elevated homocysteine levels such as vascular disease. Often these people do not receive treatment since the symptoms of these diseases are not easily recognized. Thus, by the time a person finally suffers recognizable symptoms of one of these diseases or are diagnosed, the severity of the disease may have become life-threatening.

Several embodiments of the present invention addresses this dilemma by providing means of treating and preventing conditions caused or exacerbated by, or associated with, elevated homocysteine levels. By providing the dietary supplement of several embodiments of the present method, one which has little or no side effects and is capable of addressing the causes and symptoms of conditions caused or exacerbated by, or associated with, elevated homocysteine levels, people are able to receive a treatment or a preventative for a disease or diseases from which they unknowingly suffer or are at risk from suffering.

It will be apparent to those skilled in the art that only the preferred embodiments have been described by way of exemplification and that there are various modifications which fall within the scope of this method.

### BRIEF DESCRIPTION OF THE DRAWINGS

FIG. 1 is a representation of the pathways by which homocysteine is metabolized.

FIG. 2 illustrates the results of the evaluation of dimethyl sulfone's potential activities conducted during the 2005 study (published in 2006), Kim et al, Efficacy of methylsulfonylmethane (MSM) in osteoarthritis pain of the knee: a pilot clinical trial. OsteoArthritis and Cartilage.

### DETAILED DESCRIPTION

According to several embodiments of the invention, dimethyl sulfone is provided to maintain or reduce physiological homocysteine levels. A physiological change in homocysteine levels includes a change in tissue, cell, cerebrospinal fluid blood and/or plasma levels of homocysteine.

In one embodiment, homocysteine acts as a methyl donor in the remethylation of homocysteine to methionine. Dimethyl sulfone (DMSO₂, methylsulfone, methylsulfonylmethane, MSM) is a nutritional supplement comprising sulfur, methyl groups, and oxygen. Dimethyl sulfone may be administered by multiple routes, including oral, intravenous, and topical.

In 2005, Applicant commissioned a study to determine the efficacy of dimethyl sulfone in the treatment of pain and physical function impairment due to osteoarthritis. The results of the study are published in Kim, et al, Efficacy of methylsulfonylmethane (MSM) in osteoarthritis pain of the knee: a pilot clinical trial. OsteoArthritis and Cartilage, herein incorporated by reference in its entirety. Patients were given dosage of 6 g of dimethyl sulfone (Distilled MSM microprill (OptiMSM®, Cardinal Nutrition, Vancouver, Wash.)) per day in a stepwise approach. In week 1, dosages started with 2 g/day in two divided doses for 3 days, and then increased to 4 g/day for 4 days. Week 2, increased to 6 g/day. In the control group, a placebo was administered that consisted of inert ingredients and that was indistinguishable in color, size and taste from the dimethyl sulfone. The study concluded that dimethyl sulfate administered in 3 g dosages twice a day improved symptoms of pain and physical function.

In several embodiments of the invention, a method of preventing or treating osteoarthritis is provided. In one embodiment, a daily dose of 4.5 g-20 g dimethyl sulfone (provided in a single dose or multiple doses) is provided for the prevention or treatment of diseases in which elevated homocysteine levels contribute to the cause or symptoms of the disease. Such diseases include, but are not limited to, osteoarthritis. Daily doses (single or multiple doses) of 4.5 g, 5 g, 5.5 g, 6 g, 6.5 g, 7 g, 8 g, 9 g, 10 g, and greater are provided. Doses lower than 4.5 g may be effective for certain diseases and/or for prevention or maintenance. For example, although administration of 6 g daily may be effective for treating osteoarthritis, less than about 4.5 g daily may be effective for preventing osteoarthritis or maintaining desired homocysteine levels.

In one embodiment, the invention comprises identifying subjects who have both osteoarthritis and homocysteine levels that are higher than average for that individual's subgroup (e.g., higher by at least 5%, 10%, 15%, 20%, 25%, 30%, 40%, 50%, 100%, 150%, 200% or greater) and/or exceed a desired level (e.g., 4-8 μmol/l). The patients identified as having OA with elevated homocysteine are administered at least 3 g, 4 g, 5 g, or 6 g of dimethyl sulfone per day. This dose is higher than several supplement doses of dimethyl sulfone because, in some embodiments, the dimethyl sulfone treats osteoarthritis by methylating homocysteine into methionine. Thus, in some embodiments, lower doses of dimethyl sulfone may be insufficient to serve as an appropriate methyl donor to homocysteine.

During the 2005 osteoarthritis study, described above, dimethyl sulfone's potential activities were also evaluated. Serum homocysteine, high sensitive C-reactive protein (CRP), erythrocyte sedimentation rate (ESR), and urine malondialdehyde (MDA) were measured at baseline and 12 weeks.

Homocysteine is formed in the body. As described in detail above, hyperhomocysteinemia is associated with cardiovascular disease and other conditions; and reducing homocysteine with micronutrients has been demonstrated to decrease vascular disease.

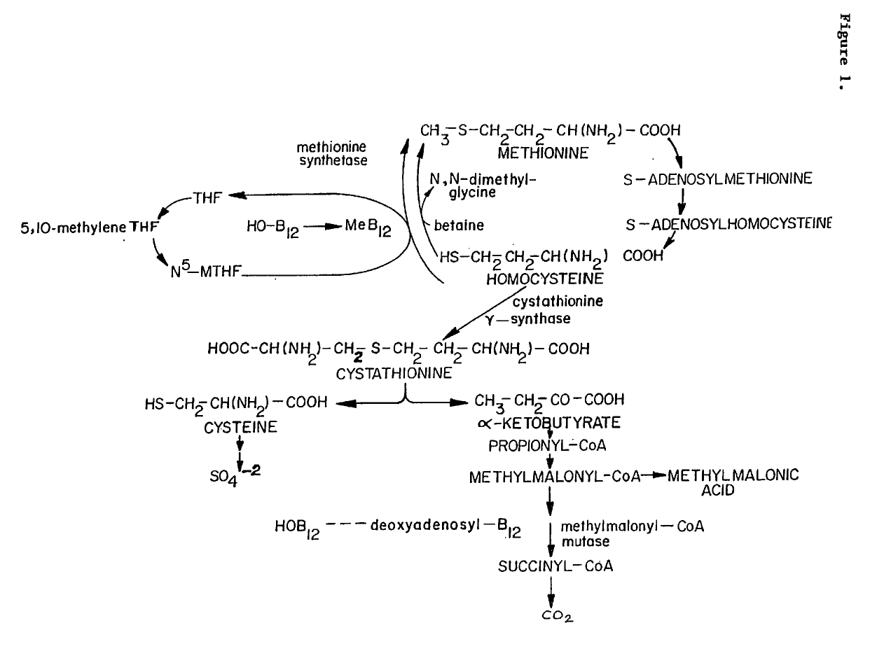

FIG. 1 shows that once homocysteine is produced it is metabolized in the body through one of two possible pathways—remethylation or transsulfuration. Remethylation is a process that utilizes folate, vitamin B-12 or betaine (trimethylglycine) to convert homocysteine back to methionine. During this process, folate acts as a methyl donor to facilitate the conversion of homocysteine to methionine.

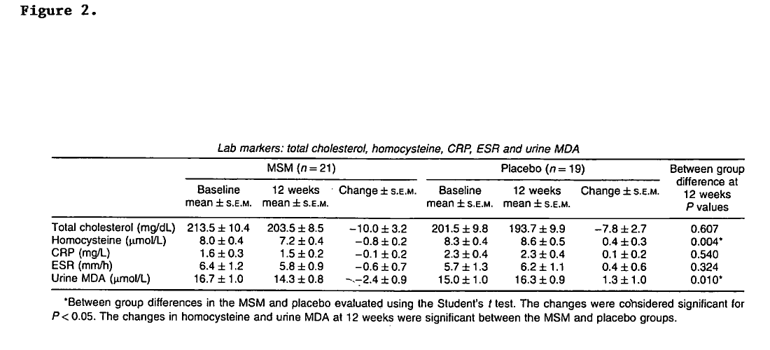

Since dimethyl sulfone is a putative methyl donor, it was believed that dimethyl sulfone may act as a co-factor in reducing homocysteine levels. FIG. 2 shows that during the 2005 osteoarthritis study homocysteine levels were significantly decreased in the group administered dimethyl sulfone. Not wishing to be bound by any particular theory, the decrease in homocysteine is believed to be due to the donation of dimethyl sulfone's two methyl groups. Folic acid and B vitamins are known to reduce hyperhomocysteinemia through similar mechanisms. Thus, in one embodiment, the invention comprises the use of a methyl donor (including but not limited to dimethyl sulfone and its related compounds) to remethylate homocysteine into methionine.

The combined decreases in homocysteine and urine MDA during the 2005 osteoarthritis study supports the role of dimethyl sulfone in metabolic processes requiring methylation, such as antioxidant capacities. Thus, in some embodiments, the invention comprises the use of dimethyl sulfone to catalyze reactions that involve methylation. In one embodiment, the invention comprises the use of dimethyl sulfone for methylating compounds other than homocysteine.

In several embodiments, dimethyl sulfone is provided alone and in combination with other nutritional ingredients including supplements, excipients, foods, beverages, food additives, and drugs specifically chosen and combined according to their biological activities.

In one embodiment, a method of treating or preventing elevated homocysteine levels and, therefore, vascular disease, includes administering a formulation including a therapeutically effective amount of dimethyl sulfone alone or in combination with nutrients or drugs intended to alter homocysteine selected from: the group consisting of the various forms of vitamin B-6 (e.g., pyridoxine), the various forms of vitamin B-12 (e.g., cyanocobalamin), the various forms of folic acid (e.g., pteroglutamic acid), betaine, andenosylmethionine, choline, acetylcysteine, and combinations thereof. Vascular Disease includes any condition that affects the circulatory system. Vascular Disease includes, bur is not limited to, vascular diseases of the arteries, veins, lymph vessels and blood disorders that affect circulation. Vascular Disease includes, bur is not limited to, cardiovascular and cerebrovascular diseases. Vascular Disease includes, bur is not limited to, peripheral arterial disease, renal artery disease, aneurysm, Raynaud's disease, varicose veins, deep vein thrombosis, blood clots, lymphedema, clotting disorders, fibromuscular dysplasia, etc.)

In some embodiments, a method of treating or preventing elevated homocysteine levels and, therefore, vascular disease, includes administering a formulation including a therapeutically effective amount of dimethyl sulfone in combination with one or more nutritional ingredients and drugs, each in a therapeutically effective amount, selected from: the various forms of vitamin B (e.g., B-1 (thiamine), B-2 (riboflavin), B-3 (niacin), B-5 (pantothenic acid); Coenzyme QIO; the various forms of vitamin E (e.g., tocopherol); amino acids such as cysteine, arginine, carnitine, 5-HTP, glutamic acid, glutamine, glycine, histidine, isoleucine, L-tyrosine, leucine, methionine, ornithine, phenylalanine, taurine, valine; anthocyanins, anthocynidins, anthocysanosides, and other antioxidant pigments; vitamin C (ascorbic acid) and its congeners; vitamin A and its congeners; carotenoids (e.g., beta-carotene, lutein, lycopene); xanthophylls; vitamin K and its congeners; vitamin D and its congeners; acetyl-L-carnitine; alanine; agae (blue-green); aloe; androstenedine; bee pollen; bee propolis; beta-glucan; beta-sitosterol; betaine HCl; the various probiotics (e.g., acidophilus); the various bioflavonoids (e.g., quercetin and rutin); biotin; black currant seed oil; boric acid; boron; bovine cartilage; bovine colostrum; brewer's yeast; bromelain; calcium D-glucarate; carnosine; cartilage; the various cetylated fattyacids (e.g., cetyl meristoleate); chitosan; chlorella; chlorophyll; chondroitin sulfate; chondroitin/glucosamine combinations; chromium; coconut oil; cod liver oil; collagen; colloidal silver; conjugated linoleic acid; copper; coral calcium; creatine monohydrate; curcumin; D-mannose; daidzein; dehydroepiandrosterone (DHEA); docosahexaenoic acid (DHA); digestive enzymes; diindolylmethane (DIM); dimethyl sulfoxide (DMSO); dimethylaminoethanol; eicosapentaenoic acid; enzymes (e.g., lactase, protease, lipase, amylase); epigallocatechin gallate (EGCG); estrogens and phytoestrogens; evening primrose oil; the various forms of iron (e.g., ferrous sulfate); fiber; fish oil; fluoride; fructo-oligosaccharides (FOS); fumaric acid; gamma linolenic acid; gamma oryzanol; gamma-amino butyric acid; garcinia cambogia; garlic; genistein; ginko and its extracts; glandular extracts (e.g., adrenal, liver, spleen, thymus, thyroid, etc.); glucaric acid; glucomannan; glucosamine; glucosamine hydrochloride; glucosamine sulfate; GTF chromium; glutamic acid; glutamine; glutathione; glycine; grape seed extract; grapefruit seed extract; green tea; green-lipped mussel; huperzine A; hydrochloric acid; hydroxycitric acid; indole-3-carbinol; inosine; inositol hexaniacinate; inositol hexaphosphate; inulin oligosaccharides; iodine; ipriflavone; kelp; lecithin; lignan; linum usitatissimum; lipoic acid; lysine; magnesium; malic acid; manganese; mannose; medium chain triglycerides; melatonin; methoxyisoflavone; milk thistle and its extracts (e.g., silymarin); molybdenum; N-acetul-glucosamine; NADH; octacosanol; oligomeric proanthocyanidins; oligosaccharides; omega-3 fatty acids; ornithine alphaketoglutarate; palm kernel oil; palm oil; pancreatic enzymes; pancreatin; papain; para-aminobenzoic acid, phosphatidyl choline; phosphatidylserine; policosanol; pregnenolone; proanthocyanidins; progesterone; propionyl-L-carnitine; protein (including soy and whey); psyllium; pyruvic acid; resveratrol; ribose; royal jelly; rutin; rye pollen; 7-KETO; saccharomyces boulardii; saccharomyces cerevisiae; selenium; shark cartilage; silica hydride; silicon; the various soy products (e.g., isoflavones); spirulina; starch blockers; strontium; sulforaphane; sulfur; thiotic acid; tyrosine; vanadium; vinacamine; vinpocetine; wheat grass; xylitol; and zinc. In some embodiments, the compounds identified above are provided in amounts ranging from 1 mg to about 100 grams. Higher or lower doses may also be provided.

According to another aspect of the method, a method of treating or preventing elevated homocysteine levels and, therefore, vascular disease, includes administering a formulation including a therapeutically effective amount of dimethyl sulfone in combination with one or more excipient selected from: silicon dioxide, stearic acid, cellulose, methylcellulose, ethylcellulose, micro crystalline cellulose, ascorbyl palmitate, crosscarmellose sodium, beeswax, benzyl alcohol, dicalcium phosphate, calcium phosphate, calcium sulfate, polyethylene glycol, locus bean products, maltodextrin, hydrogenated vegetable oil, colorants and dyes (both natural and artificial), natural and artificial flavors, sugars (e.g., glucose, fructose, sucrose, sorbitol, aspartame, high fructose corn syrup, etc.), gelatin, glycerin, fatty acid derivatives (e.g., glyceryl monostearate, etc.), hydroxypropyl methylcellulose phthalate, maltol, polyvinyl-pyrrolidone, potassium sorbate, phthalates, rice flour, rice powder, shellac, silica, talc, sodium benzoate, sodium carboxymethylcellulose, sodium lauryl sulfate, sorbitan mono-oleate, sorbitan tri-oleate, sunflower oil, titanium dioxide, and xanthan gum.

In some embodiments, the nutritional supplement, excipient, food, beverage, food additive, or drug component is selected as an ingredient in the administered composition for its ability to treat elevated homocysteine levels or to prevent elevated homocysteine levels. In several embodiments, dimethyl sulfone is provided in combination with another one or more other compounds to synergistically maintain or reduce homocysteine levels. In other embodiments, dimethyl sulfone is provided in combination with another one or more other compounds, wherein the other compounds do not affect homocysteine levels. Said other compounds, for example, may be used as a preservative, colorant, flavorant, delivery agent, etc. Said other compounds may also be therapeutic for other diseases or illnesses.

In one embodiment of the invention, a measurement of the level of homocysteine in a subject is made. In one embodiment, this is done by one of several different analytical methods: capillary gas chromography—mass spectrometry (GC-MS), liquid chromatography electrospray tandem mass spectrometry (LC-MS-MS), high-pressure liquid chromatography (HPLC) with photometric detection, HPLC with fluorometric detection, HPLC with electrochemical detection, or immunoassay. Other methods may also be used.

Homocysteine levels are currently measured through the use of several different analytical methods: capillary gas chromography—mass spectrometry (GC-MS), liquid chromatography electrospray tandem mass spectrometry (LC-MS-MS), high-pressure liquid chromatography (HPLC) with photometric detection, HPLC with fluorometric detection, HPLC with electrochemical detection, and immunoassay.

The homocysteine concentration frequently correlates inversely with the folate concentration, since folate is a cofactor for the enzyme MTHFR that breaks down homocysteine. A detection of folate level in addition to the homocysteine level is therefore diagnostically appropriate. Since folate is mainly present and takes effect in the erythrocytes (approx. 98%), the detection of the erthyrocytic folate or total folate is more conclusive than the usual detection of the plasma folate or serum folate.

According to one embodiment, the homocysteine level is analyzed relative to a standard, for example, 10% above a normal level for this person or the average level of the patient's cohort/subpopulation. If the “normal level” is 12 μmol/l, then an elevated level will be defined as 10% above the normal level, i.e., 13.2 μmol/l.

If, in one embodiment, the measured homocysteine level exceeds 13.2 μmol/l then an elevated level of homocysteine in the person is said to be detected. This act of detecting will vary from person to person depending on the normal level of homocysteine for that person. In other embodiments, identifying a person with elevated homocysteine comprises identifying a person having a homocysteine level that is greater than 6 μmol/l, 7 μmol/l, 8 μmol/l, 9 μmol/l, 10 μmol/l, 11 μmol/l, 12 μmol/l, 13 μmol/l, 14 μmol/l, 15 μmol/l, 16 μmol/l, 17 μmol/l, 18 μmol/l, 19 μmol/l, or 20 μmol/l. In yet other embodiments, identifying a person with elevated homocysteine comprises identifying a person having a homocysteine level that is greater than 10%-20%, 20%-40%, 40-60%, 60-80%, 100%, 150% or 200% his or her baseline homocysteine level, wherein the baseline homocysteine level is measured prior to onset or progression of a disease state. In further embodiments, identifying a person with elevated homocysteine comprises identifying a person having a homocysteine level that is greater than 10%-20%, 20%-40%, 40-60%, 60-80%, 100%, 150% or 200% the homocysteine level of that person's cohort/subpopulation. For example, if the average homocysteine level for healthy males aged 18-35 is between about 6 μmol, a 20-year old male having a homocysteine level higher than 6.6 μmol/l would be identified as having an elevated homocysteine level.

In several embodiments of the invention, homocysteine levels are reduced by at least 10%-20%, 20%-40%, 40-60%, 60-80%, 100%, 150%, 2-fold, 5-fold, or 10-fold post administration of dimethyl sulfone.

According to one embodiment, in response to detecting an elevated level of homocysteine in the person, dimethyl sulfone is administered orally in the range of 1 mg up to 50,000 mg (e.g., 2 g, 3 g, 4 g, 5 g, 6 g, 7 g, 8 g, 9 g, 10 g or higher). The dimethyl sulfone may be combined with other ingredients, as discussed above, including, for example, vitamin B-6, vitamin B-12, folic acid, and/or betaine. Formulations comprising 1-10 mgs folic acid, 1-10 mgs vitamin B12, 50-200 mg of vitamin B6 and 1-10 grams of dimethyl sulfone are provided in some embodiments. Formulations comprising vitamin K and dimethyl sulfone are provided in other embodiments to maintain and/or reduce homocysteine levels.

In another preferred embodiment of the method, dimethyl sulfone is administered intravenously in the range of 1,000 mg up to 150,000 mg per day (e.g., 2 g, 3 g, 4 g, 5 g, 6 g, 7 g, 8 g, 9 g, 10 g or higher). In some embodiments, the daily dosage does not exceed 10 g, 20 g or 50 g per day.

According to some embodiments, the compositions described herein are prepared in a caplet dosage for, capsules, tablets, powders, pastes, liquids and similar dosage forms. Solid dosage forms for oral administration include caplets, capsules, tablets, pills, powders, and granules.

Liquid dosage forms for oral administration include pharmaceutically acceptable emulsions, solutions, suspensions, syrups, and elixirs.

Oral dosages dimethyl sulfone, alone or combined with other ingredients, may also be incorporated and administered in food and beverage products.

In one embodiment, the compositions are administered in spaced dosages throughout the day, for example, administered every twelve hours, so as to maintain the level of active ingredients in the system of the host. In other embodiments, a time-release formulation of dimethyl sulfone is provided.

The following examples describe non-limiting embodiments of the invention.

### Example 1

In a study directed by Applicant or its related affiliate, and as reported in Kim et al (published in 2006), herein incorporated by reference in its entirety, the effects of dimethyl sulfone on OA of the knee were evaluated. Qualified patients (n=50) were as-signed to MSM (n=25) or placebo (n=25) in a 12-week randomized, double-blind, placebo-controlled trial using computer-generated random numbers (FIG. 1).

At week 1, subjects were administered 2 g/day in two divided doses for 3 days, and then increased to 4 g/day for 4 days. At week 2, this was increased to 6 g/day. Distilled MSM microprill (OptiMSM®, Cardinal Nutrition, Vancouver, Wash.) in 1 g caps was used. Purity of dimethyl sulfone was confirmed to be 99.9% by high-resolution gas chromatography. DMSO content was <0.05%. The placebo consisted of inert ingredients and was indistinguishable in color, size and taste compared to the dimethyl sulfone. Test materials were certified to be free of microbiological contamination. In this trial, dimethyl sulfone at 3 g twice a day for 12 weeks produced improvement in two of the three WOMAC subscales, pain and physical function. Thus, according to one embodiment of the invention, dimethyl sulfone is administered to treat OA, wherein dimethyl sulfone is provided in a dose of about 6 g/day. In other embodiments, dimethyl sulfone is administered in doses of 1-2, 2-4, 4-6, 6-8, 8-10, or greater than 10 g/day. In several embodiments, these doses may also be effective for other types of arthritis and inflammatory conditions. In several embodiments, the invention comprises a method of reducing pain and/or increasing physical function by administering dimethyl sulfone.

### Example 2

In a study directed by Applicant or its related affiliate in 2008, six participants received three doses of dimethyl sulfone at three acute test visits spaced one week apart. Subjects were monitored for four hours post dose, with pharmacokinetic (PK) and phamacodynamic blood draws at 0, 45, 90, 135, 180 and 240 minutes post dosing. Subjects also provided a 24-hour post-dose urine collection for assay of dimethyl sulfone and sulfate levels.

Serum dimethyl sulfone levels displayed the rise and fall pattern consistent with fairly rapid absorption from the upper gastrointestinal tract (within an hour), followed by slower elimination from the bloodstream (over the course of one or two days). Rough estimates have been obtained for the half times of absorption (½ hour) and elimination (8 hours).

In some embodiments, administration of dimethyl sulfone results in carry-over of dimethyl sulfone from one visit to the next. Thus, terminal elimination half-life, according to some embodiments may be higher than 10 or 12 hours.

Dimethyl sulfone, in one embodiment, appears to dose-dependently alter the sulfur compartment of at least the plasma. For example, acute oral dosing with dimethyl sulfone might, at increasingly higher doses, alter the compartmentalization and metabolism of sulfur, and transsulfuration pathways, resulting in increased apparent sulfur retention (via reduced urine sulfate output) and reduced circulating homocysteine concentrations. Thus, in one embodiment, the invention comprises a method for increasing sulfur retention and/or reducing circulating homocysteine by the administration of dimethyl sulfone.

What is claimed is:

1. A method for reducing elevated levels of homocysteine in a subject, the method comprising:
measuring a homocysteine level in a subject;
detecting an elevated level of homocysteine in the subject; and
responsive to said step of detecting, administering dimethyl sulfone to said subject,
wherein said dimethyl sulfone is administered in a daily dosage of dimethyl sulfone,
wherein said daily dosage of dimethyl sulfone is greater than 3 grams, and
wherein said dimethyl sulfone reduces the elevated level of homocysteine in the subject.

2. The method of claim 1, wherein dimethyl sulfone is administered in a daily dosage of dimethyl sulfone of between 6 grams to 10 grams.

3. The method of claim 1, wherein said daily dosage is administered in a single dose.

4. The method of claim 1, wherein said daily dosage is administered orally.

5. The method of claim 1, wherein said daily dosage is administered intravenously.

6. The method of claim 1, wherein said dimethyl sulfone reduces the elevated level of homocysteine in the subject by an amount sufficient to reduce pain in said subject.

7. The method of claim 1, wherein said dimethyl sulfone reduces the elevated level of homocysteine in the subject by an amount sufficient to improve physical function in said subject.

8. The method of claim 1, wherein said dimethyl sulfone reduces the elevated level of homocysteine in the subject by at least 10%.

9. The method of claim 1, wherein said dimethyl sulfone reduces the elevated level of homocysteine in the subject by 25-50%.

10. The method of claim 1, wherein said subject has osteoarthritis, and wherein said dimethyl sulfone.

11. The method of claim 1, further comprising administering a second compound to said subject.

12. The method of claim 11, wherein said second ingredient maintains or reduces the homocysteine level.
